(19) United States
(12) Patent Application Publication
(10) Pub. No.: US 2004/0176982 A1
(43) Pub. Date: Sep. 9, 2004

(54) Method of providing health care services

(76) Inventors: Timothy M. Kilgore, Reno, NV (US); David L. Haas, Reno, NV (US)

Correspondence Address: Michael W. Starkweather, Suite 600, 8 East Broadway, Salt Lake City, UT, 84111, US

(21) Appl. No.: 10/790,500

(22) Filed: Mar. 1, 2004

Related U.S. Application Data
(60) Provisional application No. 60/451,448, filed on Mar. 3, 2003.

(51) Int. Cl. A61B 5/00; G06F 17/60

(52) U.S. Cl. 705/2; 705/35

(57) ABSTRACT

A system and method for providing health care services including a health care insurance plan to one or more associations are presented. The invention includes a parent management company organized to manage general operations, provide a health care service, and provide an operational process. The parent management company organizes an association health component as a subsidiary of the association and trains the personnel of the association health component. The parent management company also provides the services of a wellness component, a risk management component, a financial services component, and a data management component. A managing general agent licenses the operational process and manages the operational process for the association health component. The association health component may franchise the health care service and the operational process to additional associations and association health components. The invention further provides access to the services through an Internet component. In one embodiment, the invention also provides a point of service component with identification and billing services.

### CROSS-REFERENCES TO RELATED APPLICATIONS

This application is a continuation-in-part of and claims priority to U.S. Provisional Patent Application No. 60/451,448 entitled “A METHOD OF PROVIDING HEALTH CARE BENEFITS” and filed on Mar. 3, 2003 for Timothy M. Kilgore and David L. Haas, which is incorporated herein by reference.

### BACKGROUND OF THE INVENTION

1. Field of the Invention

The present invention relates generally to a system and method of providing employer based health care coverage. Specifically, the present illustrated embodiment(s) involve(s) the use of customized health care insurance plans which utilize a unique underwriting process, which are administered by an association health component such as a captive association health plan company, and which involves an unique associations health care services formation process.

2. Background of the Illustrated Embodiment(s)

Health care service in the United States has been designed and employed to provide treatment to persons who have become ill or injured. The cost of obtaining health care insurance plans (“HCIP”) is often prohibitively high, and insurance rates are increasing by as much as 15% annually. Small employers and small businesses are often unable to obtain affordable HCIP because of small subscriber bases. As a result, as many as 41 million Americans including many that are employed do not have health insurance.

In addition to increasingly costs, many HCIP are inadequate in providing the types of services that many people require and want. Prevention and proactive wellness programs have been severely lacking. Many current plans have high deductibles or have limited coverage for wellness programs and alternative care. For many small and medium businesses, offering a variety of HCIP coverage options including prevention and proactive care to their employees has become difficult, if not impossible, because of the costs and administrative complexities. Small and medium business have also had limited access to risk management services, data management services and HCIP financial services.

The following United States patents, and patent applications, are herein incorporated by reference for their supporting teachings:

U.S. patent application Ser. No. 2002/0087444 discloses techniques and an apparatus for managing contributions to an accruable health spending account in an employer sponsored plan offering a member an employee-funded defined contribution, at least one insurance premium option and the ability to specify an allocation of the defined contribution for payment of option premiums;

U.S. patent application Ser. No. 2002/0049617 discloses a system and method of providing benefits which includes identifying at least one price for each of plurality of line items within a benefit category, and offering the line items for purchase by the individual;

U.S. patent application Ser. No. 2002/0152097 discloses a method for benefits administration, which is comprised of a Health Care Account and an Umbrella Account. Through financial incentives, benefit plan beneficiaries are encouraged to spend funds in the Health Care Account and the Umbrella Account so that, at the end of the year, unspent funds are distributed back to the beneficiaries;

U.S. patent application Ser. No. 2001/0037214 discloses a method and system that incentives an employee to forego coverage under one or more health care plans maintained by the employee's employer. An employee eligible for health care coverage under another plan or plans not sponsored by the employer is given the option to elect between coverages;

U.S. Pat. No. 5,301,105 discloses a fully integrated and comprehensive health care system that includes the integrated interconnection and interaction of the patient, health care provider, bank or other financial institution, insurance company, utilization reviewer and employer; and

U.S. patent application Ser. No. 2002/0069090 discloses a system and method for the employee benefits industry including a client management module for providing a broker with one or more tools for generating an employer record and employee record.

It is believed that all of the listed patents do not anticipate or make obvious the disclosed preferred embodiment(s).

### PROBLEMS WITH THE PRIOR ART

Small employers and small business have sought to obtain affordable HCIP through associations by forming association health plans (“AHP”) to increase the collective subscriber base and to reduce administrative costs for all participating association members. Associations may include trade groups and local business organizations. Association members obtain HCIPs through the AHP as participants. AHPs have consolidated the subscriber bases and reduced administrative costs for association members.

Unfortunately, associations often lack the geographic breadth required to provide adequate service wherever association members are located. Associations may also lack subscriber bases sufficient large to contract for medical and administrative services at the lowest costs. AHPs may further lack the size required to provide cost-effective wellness and Internet access services. In addition, AHPs may be too small to afford risk management services to reduce the liability risk of AHP participants or to purchase the financial services needed to manage the AHP's reserves.

What is needed is a system and method for providing AHPs with larger subscriber bases, centralized management, extended financial services, risk management, and wellness programs. In addition, a system a method is needed to provide AHPs with service in extended geographic locations and with Internet and integrated point of service data capabilities.

### SUMMARY OF THE ILLUSTRATED EMBODIMENT(S)

The present invention has been developed in response to the present state of the art, and in particular, in response to the problems and needs in the art that have not yet been fully solved by currently available systems and methods of providing health care services. Accordingly, the present invention has been developed to provide a system and method for providing health care services that overcome many or all of the above-discussed shortcomings in the art.

A system for providing health care services is presented. The system includes an association, a parent management company, an association health component, a wellness component, a risk management component, a financial services component, a data management component, and a managing general agent.

The association includes a plurality of members. In one embodiment, the parent management company recruits the association as a client. The parent management company organizes the association health component (“AHC”). The AHC may be an association health plan company (“AHPC”). In one embodiment, the AHC is a captive AHPC. In an alternate embodiment, the AHC is a worker's compensation company (“WCC”). The AHC includes a plurality of members. Members may be individuals, groups, and businesses.

In one embodiment, the parent management company manages the AHC for the association. In an alternate embodiment, the parent management company provides management support for the AHC to the association. In a certain embodiment, the association franchises the operational process of the AHC from the parent management company. The association may further franchise the operational process of the AHC to one or more additional associations. The parent management company trains the sales and administrative personal of the AHC to market and administer one or more health care services (“HCS”).

The parent management company provides the AHC with the HCS. In one embodiment, the HCS is purchased from an independent service provider. In an alternate embodiment, the HCS is purchased directly from the parent management company. The parent management company further provides the wellness component to the AHC. The wellness component provides preventative care and wellness education to the AHC. The wellness component may provide traditional wellness services such as immunizations. In a certain embodiment, the wellness component also provides non-traditional wellness services such as aroma-therapy and massage therapy.

The risk management component provides risk management services to the AHC under the direction of the parent management company. The risk management component may provide risk reduction training and a risk reduction program to the AHC members. The financial services component provides financial services to the association health plan company under the direction of the parent management company. The financial services component may support underwriting health care services.

The data management component captures patient and employer data. In one embodiment, the data management component captures data from the AHC. In an alternate embodiment, the data management component captures data from a HCS service provider. The data management component may also capture data from an AHC member.

The managing general agent licenses the operational process for the AHC. In addition, the managing general agent may manage the operational process for the AHC. The managing general agent may be an employee of the parent management company. In an alternate embodiment, the managing general agent is an employee of the AHC. In a certain embodiment, the managing general agent is an independent contractor. In one embodiment, the AHC franchises the HCS and the operational process to one or more additional associations.

A method for providing health care is also presented. The method in the disclosed embodiments substantially includes the steps necessary to carry out the functions presented above with respect to the operation of the described system. The method includes recruiting an association, organizing an AHC, providing the AHC with a HCS, an operational process, and a managing general agent, managing the HCS, training sales and administrative personal, providing a wellness service, providing a risk management service, providing a financial service, and providing a data management service.

The method recruits an association to a health care system. The method further organizes an AHC as a subsidiary of the association. In one embodiment, the method organizes the AHC as a captive AHPC. In a certain embodiment, the association includes an existing AHC. The method provides a HCS and an operational process for managing the HCS to the AHC. In one embodiment, the parent operation company manages the HCS for the AHC. In addition, the method provides training for the sales and administrative personnel of the AHC. The method further provides a wellness service, a financial service, a managing general agent, and a data management service to the AHC.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

The present invention organizes an AHC for an association and provides a HCS to the AHC along with a wellness service, a financial service, a risk management service, a managing general agent, and a data management service. The present invention further licenses and franchises an operational process for the AHC and HCS. These features and advantages of the present invention will become more fully apparent from the following description and appended claims, or may be learned by the practice of the invention as set forth hereinafter.

### DETAILED DESCRIPTION OF THE ILLUSTRATED EMBODIMENT(S)

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

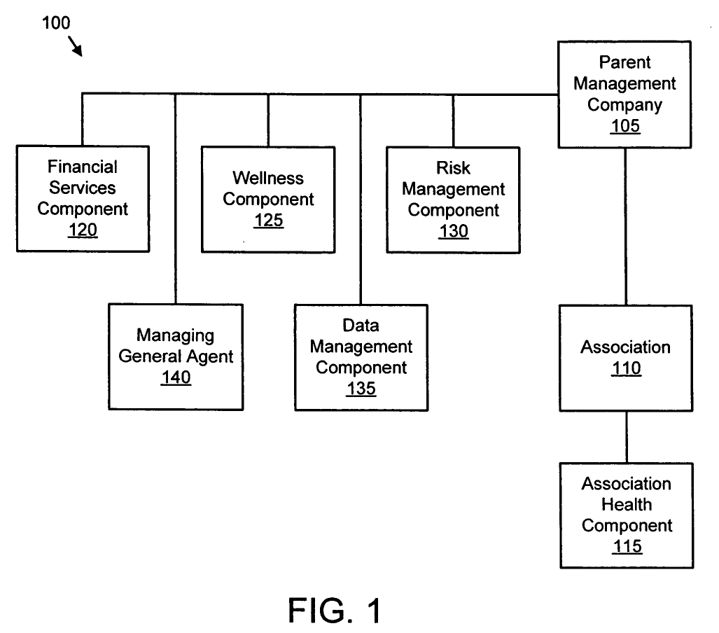

FIG. 1 is a block diagram illustrating one embodiment of a health care system in accordance with the present invention;

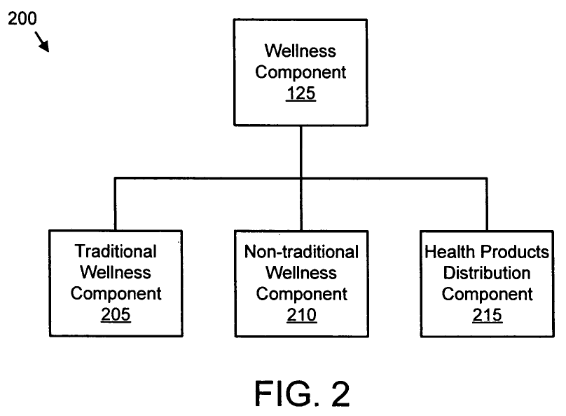

FIG. 2 is a block diagram illustrating one embodiment of a wellness organization of the present invention;

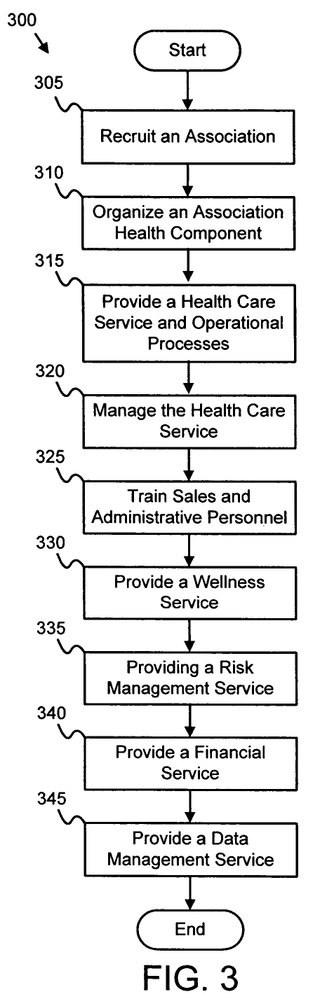

FIG. 3 is a flow chart illustrating one embodiment of a health care method in accordance with the present invention;

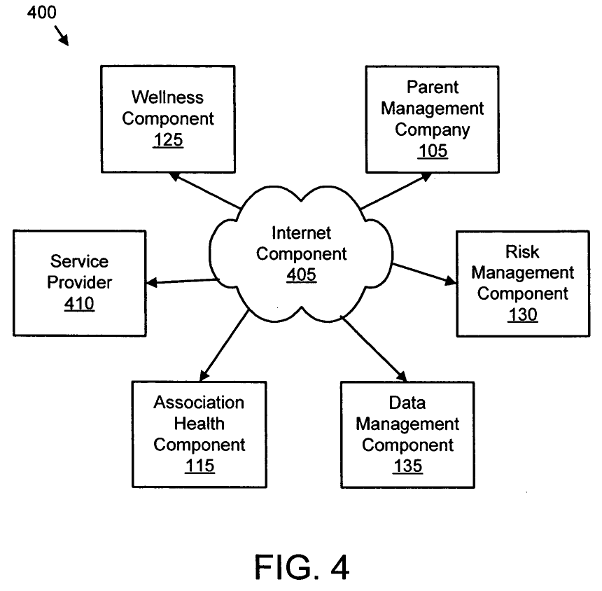

FIG. 4 is a block diagram illustrating one embodiment of an Internet information system of the present invention;

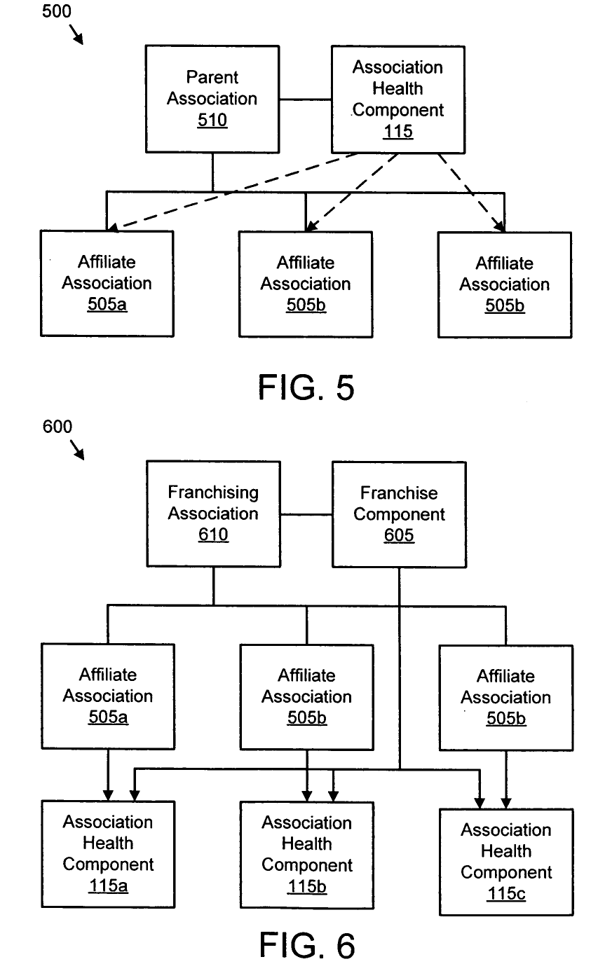

FIG. 5 is a block diagram depicting one embodiment of a multi-association health care system of the present invention; and

FIG. 6 is a block diagram illustrating one embodiment of a franchised health care system in accordance with the present invention.

### DETAILED DESCRIPTION OF THE ILLUSTRATED EMBODIMENT(S)

For the purpose of promoting an understanding of some of the principles of the illustrated embodiment(s), reference will now be made to exemplary embodiment(s) that are illustrated in the figures, and specific language will be used to describe the same. It will nevertheless be understood that no limitation of the scope of the claims is thereby intended. Any alterations and further modifications of the inventive features illustrated herein, and any additional applications of these principles, which would be considered within the scope of this invention.

The present invention generally relates to a system and method of providing health care services through an association model. More specifically, and by way of illustrative purposes only, the illustrative embodiment specifically relates to a method of providing health care services which may: target associations that create commonality among members to achieve a reduction in benefit rates, provide wellness and risk management components or programs, provide outsourcing of major contracts to achieve reduced costs, establish industry alliances to achieve a large profitably base, and reduce long-term health care costs.

FIG. 1 is a block diagram illustrating one embodiment of a health care system 100 in accordance with the present invention. The health care system 100 provides health care services (“HCS”) including risk management and wellness services to one or more associations. The health care system 100 includes a parent management company 105, an association 110, an association health component (“AHC”) 115, a financial services component 120, a wellness component 125, a risk management component 130, a data management component 135, and a managing general agent 140. Although the health care system 100 is depicted with one association 110 and one AHC 115, any number of associations 110 and any number of AHCs 115 may be included.

The association 110 includes a plurality of members. In one embodiment, the association 110 is a trade group. In an alternate embodiment, the association 110 has geographic based membership. In a certain embodiment, association 110 members are individuals. Association 110 members may also be groups and businesses. The parent management company 105 recruits the association 110 to join the health care system 100.

The parent management company 105 organizes the AHC 115 as a subsidiary of the association 110. In one embodiment, the parent management company 105 provides legal, managerial, and HCS expertise. The AHC 115 may be an association health plan company (“AHPC”). In one embodiment, the AHC 115 is a captive AHPC. The captive AHPC is controlled by the association 110 and may also be owned by the association 110. The captive AHPC may be a rental captive. A rental captive enters into a contractual relationship with the association 110 and the association 110 members to provide services such as HCS. The captive AHPC may also be an agency captive. The agency captive is owned by service provider such as an insurance agency or a brokerage and provides the service provider's services. In one embodiment, the captive AHPC is a pure captive and only insures the risks of the association 110 and the association's 110 members. In an alternate embodiment, the AHC 115 is a worker's compensation company (“WCC”).

In one embodiment, the parent management company 105 manages the AHC 115 for the association 110. In an alternate embodiment, the parent management company 105 provides management support for the AHC 115 to the association 110 and the AHC 115. In a certain embodiment, the association 110 licenses an operational process of the AHC 115 from the parent management company 105. In an alternate embodiment, the association 110 franchises the operational process of the AHC 115 from the parent management company 105. The association 110 may further franchise the operational process of the AHC 115 to one or more additional associations.

The parent management company 105 provides the AHC 115 with the HCS. The HCS may include but is not limited to: primary medical care, specialty medical care such as oncology and cardiac specialties, chiropractic care, hospitalization, ambulatory care, home health care such as in-home residence care, preventative care such as annual physicals and immunizations, discounted pharmaceutical care, mental health care, diagnostic services such as laboratory testing, maternity care, pre-natal care, and alternative medical care.

The HCS may be a health care insurance plan (“HCIP”). In one embodiment, the HCS includes one or more different HCS options. Each HCS option may have a deductible schedule, a cost schedule, and a set of services. The HCS options may be tailored to the requirements of the association 110 and each association 110 member. In a certain embodiment, the HCS is available in multiple geographies, regardless of the geographic limitations of the sponsoring association 110. In one embodiment, the HCS is organized as a preferred provider organization service. In alternate embodiment, the HCS is organized as a managed care service.

In one embodiment, the HCS is purchased from an independent service provider. In an alternate embodiment, the HCS is purchased directly from the parent management company 105. The parent management company 105 further provides the wellness component 125 to the AHC 115. The wellness component 125 is organized under the management of the parent management company 105 to provide preventative care and wellness education to the AHC 115. The wellness component 125 may provide traditional wellness services such as immunizations. In a certain embodiment, the wellness component 125 provides non-traditional wellness services such as aroma-therapy and massage therapy.

The risk management component 130 provides risk management services to the AHC 115 under the direction of the parent management company 105. The risk management component 130 may provide risk reduction training and a risk reduction program to the association 110. In one embodiment, the risk management component 130 is owned by the parent management company 105. In an alternate embodiment, the risk management company 130 is an independent service provider contracted to provide risk management services.

In one embodiment, the risk management component 130 includes a substance abuse component, a risk management education component, and a tracking component. The substance abuse component may provide substance abuse screening and substance abuse policies to the AHC 115 and the AHC 115 members. The risk management education component may provide one or more risk management education and risk reduction programs to the AHC 115 members. In addition, the tracking component may track work and health related incidents. In one embodiment, the tracking component works with the data management component 135.

The financial services component 120 provides financial services to the AHC 115 under the direction of the parent management company 105. The financial services component 120 may support underwriting the HCS. In addition, the financial services component 120 may support financing the HCS. In a certain embodiment, the financial services component provides a stop loss services to the AHC 115. In one embodiment, the financial services component 120 is owned by the parent management company 105. In an alternate embodiment, the financial services component 120 is an independent service provider contracted to provide financial services.

The data management component 135 captures patient and employer data. The data may be provided to an actuary to determine prices for the HCS. In addition, the data may be used to determine underwriting and financing strategies for the AHC 115. The managing general agent 140 licenses the operational process for the AHC 115. In addition, the managing general agent 140 may manage the operational process for the AHC 115. The managing general agent 140 may be an employee of the parent management company 105. In an alternate embodiment, the managing general agent 140 is an employee of the AHC 115. In a certain embodiment, the managing general agent is an independent contractor under contract to the AHC 115.

In one embodiment, the health care system 100 combines the purchasing power of a plurality of associations 110 to increase the subscriber base for purchasing the HCS in order to negotiate lower the HCS costs. In a certain embodiment, the economies of scale for managing health care services for one or more associations 110 also reduces the administrative costs of the HCS.

FIG. 2 is a block diagram illustrating one embodiment of a wellness organization 200 of the present invention. The wellness organization 200 provides association 110 members with services. In the depicted embodiment, the wellness organization 200 includes a wellness component 125, a traditional wellness component 205, a non-traditional wellness component 210, and a health products distribution component 215.

In the depicted embodiment, the traditional wellness component 205, the non-traditional wellness component 210, and the health products distribution component 215 are organized under the direction of the wellness component 125. In an alternate embodiment, the parent management company 105 contracts for the traditional wellness component 205, the non-traditional wellness component 210, and the health products distribution component 215. In one embodiment, the traditional wellness component 205 provides a traditionally accepted HCS such as well-baby checks, immunizations, and annual physicals. The non-traditional wellness component 210 provides wellness services not traditionally offered by a HCIP. In a certain embodiment, the non-traditional wellness component 210 is provided as an option to the AHC 115. The non-traditional wellness component 210 include but is not limited to herbal supplements, vitamins, aroma-therapy, herbal medicine, juice therapy, acupuncture, sound therapy.

The health products distribution component 215 provides other health related services to the AHC 115 and association 110 members. In one embodiment, the health products distribution component 215 retails health related clothing and equipment. In an alternate embodiment, the health products distribution component 215 provides discounts at health related businesses including but not limited to gyms, retail stores, and spas. In a certain embodiment, the health products distribution component 215 is provided as an option to the AHC 115 and to the association 110 members. The wellness organization 200 provides association 110 members with services beyond what is practical for a single association 110.

FIG. 3 is a flow chart illustrating one embodiment of a health care method 300 in accordance with the present invention. The health care method 300 organizes an AHC 115 and provides the AHC 115 with a HCS. Although for purposes of clarity the health care method 300 is depicted in a certain sequential order, execution may be conducted in parallel and not necessarily in the depicted order.

The health care method 300 recruits 305 an association 110 and organizes 310 an AHC 115. In a certain embodiment, the AHC 115 is a subsidiary of the association 110. In one embodiment, the AHC 115 is an AHPC. In an alternate embodiment, the AHC 115 is a captive AHPC. In a certain embodiment, the AHC 115 is a WCC.

The health care method 300 provides 315 a HCS, an operational process for the HCS and a managing general agent 140. In addition, the health care method 300 manages 320 the HCS. In one embodiment, a parent management company 105 manages 320 the HCS. In an alternate embodiment, the AHC 115 manages 320 the HCS with the support of the parent management company 105. The AHC 115 may license the operational process for managing the HCS from the parent management company 105. In addition, the AHC 115 may also license the HCS and the operational process as a franchisee. The managing general agent 140 licenses and manages the operational process for the AHC 115. In a certain embodiment, the AHC 115 franchises the HCS and the operational process to one or more additional associations. For example, a national association may franchise the HCS and the operational process to one or more regional affiliate associations.

In one embodiment, the health care method 300 trains 325 the sales force and the administrative personal of the AHC 115. The health care method 300 provides 330 a wellness service through a wellness component 125. In addition, the health care method 300 provides 335 a risk management service and provides 340 a financial service. The health care method 300 further provides 345 a data management service. The health care method 300 provides a HCS to an association 110 by organizing 310 and managing 320 an AHC 115.

FIG. 4 is a block diagram illustrating one embodiment of an Internet information system 400 of the present invention. The Internet information system 400 provides online access to one or more services. The Internet information system 400 includes a wellness component 125, a parent management company 105, an Internet Component 405, a service provider 410, an AHC 115, a data management component 135, and a risk management component 130. Although for simplicity the Internet information system 400 is depicted with one service provider 410 and one AHC 115, any number of service providers 410 and AHCs 115 may be included.

The Internet component 405 provides a gateway for the association 110, the AHC 115, and the association 110 members to access services. A user may access the services of the wellness component 125, the parent management company 105, one or more service providers 410, the AHC 115, and the risk management component 130 through the Internet component 405. In one embodiment, the Internet component 405 includes components created by the parent management company 105. In a certain embodiment, the Internet component contains components from the wellness component 125, the parent management company 105, one or more service providers 410, one or more AHCs 115, and the risk management component 130.

In one embodiment, the Internet component 405 collects data for the data management component 135. The Internet component 405 may also provide data from the data management component 135. In a certain embodiment, Internet component 405 accesses the services of the financial services component 120 and the managing general agent 140 through the parent management company 105. The Internet information system 400 provides online access to services within and associated with the health care system 100.

In one embodiment, the Internet component 405 includes a point of service component. The point of service component provides identification and billing services on the site of the service provider 410. In one embodiment, each AHC 115 member is issued a data card. The data card may store patient data and identify the AHC 115 member. In a certain embodiment, the data card is a smart card. In an alternate embodiment, the data card is a magnetic strip card.

FIG. 5 is a block diagram depicting one embodiment of a multi-association health care system 500 of the present invention. The multi-association health care system 500 provides a HCS to one or more associations 110. The multi-association health care system 500 includes a parent association 510, an AHC 115, and one or more affiliate associations 505. In one embodiment, the parent association 510 is the association 110 of FIG. 1.

The parent association 510 provides the HCS through the AHC 115 to the affiliate associations 505. In one embodiment, the AHC 115 customizes the HCS for each affiliate association 505. In an alternate embodiment, the AHC 115 provides the same HCS for each affiliate association 505. The AHC 115 also provides the financial services component 125, the wellness component 130, the risk management component 130, the data management component 135, and the managing general agent 140 as described in FIG. 1. The multi-association health care system 500 provides the HCS through the parent association 510 to one or more affiliate associations 505.

FIG. 6 is a block diagram illustrating one embodiment of a franchised health care system 600 in accordance with the present invention. The franchised health care system 600 franchises an AHC 115 to one or more affiliate associations 505. The franchised health care system 600 includes a franchise component 605, a franchising association 610, one or more affiliate associations 505 and one or more AHCs 115.

The franchising association 610 organizes a franchise component 605. In one embodiment, the franchising association 610 is the association 110 as described in FIG. 1. In a certain embodiment, the franchise component 605 is an AHC 115 as described in FIG. 1. The franchise component 605 organizes the AHC 115 for the affiliate associations 505 and franchises the AHC 115, a HCS, and an operational process to each affiliate association 505. Each AHC 115 may operate as a subsidiary of the affiliate association 505. In addition, each AHC 115 provides the HCS to the affiliate association 505 members as a franchise of the franchise component 605. The franchised health care system 600 provides the HCS through one or more franchised AHCs 115.

Variations of the Illustrated Embodiment(s)

The present invention organizes an AHC 115 for an association and provides an HCS to the AHC 115 along with a wellness component 125, a financial services component 120, a risk management component 130, a managing general agent 140, and a data management component 135. The present invention may further license and franchise an operational process for the AHC 115 and HCS. It is understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements.

For example, although the illustrative embodiment(s) has discussed the use of standard health care services, there are many forms of alternate policies that may be adopted into the present model, such as including nontraditional wellness services or unconventional medical practices, according to the established needs of particular industries, companies, or trades.

Thus, while the present invention has been shown in the drawings and fully described above with particularity and detail in connection with what is presently deemed to be the most practical and preferred embodiment(s) of the invention, it will be apparent to those of ordinary skill in the art that numerous modifications, including, but not limited to, variations in dependency of components, and function and manner of operation may be made, without departing from the principles and concepts of the invention as set forth in the claims.

What is claimed is:

1. A system for providing health care benefits, comprising:
an association with a plurality of members;
a parent management company organized to manage a health care service for the association;
an association health component, organized by the parent management company as a subsidiary of the association and provided with the health care service and an operational process by the parent management company, the sales and administrative personnel of the association health component trained by the parent management company;
a wellness component under the direction of the parent management company to provide a preventative care and wellness education service to the association health component;
a risk management component providing a risk management service to the association health component under the direction of the parent management company;
a financial services component providing a financial service to the association health component under the direction of the parent management company;
a data management component organized to capture patient and employer data under the direction of the parent management company; and
a managing general agent who licenses the operational process and manages the operational process for the association health component.

2. The system of claim 1, wherein the association health component contracts the health care service from a service provider in coordination with the parent management company.

3. The system of claim 1, wherein the association health component operates as an association health plan company.

4. The system of claim 1, wherein the association health component operates as a captive association health plan company.

5. The system of claim 1, wherein the association health component is a workers compensation company and the health care service is a workers compensation insurance.

6. The system of claim 1, the risk management component further comprising:
a substance abuse component providing substance abuse screening and substance abuse policies;
a risk management education component providing risk management education programs; and
a tracking component organized to track work and health related incidents.

7. The system of claim 1, further comprising a point of service component configured to provide identification and billing services on the site of a service provider.

8. The system of claim 7, wherein the point of service component comprises a data card configured to store patient data.

9. The system of claim 8, wherein the data card is a smart card.

10. The system of claim 8, wherein the data card is a magnetic strip card.

11. The system of claim 1, the wellness component further comprising a health products distribution component distributing health products to the association and the association members.

12. The system of claim 1, further comprising an Internet component configured to allow access to the data management component, the wellness component, the risk management component, a heath care service provider, the association health component, and the parent management company.

13. The system of claim 1, further comprising a sales force organized to sell the health care service.

14. A method of providing health care benefits, the method comprising:
recruiting an association with a plurality of members;
organizing an association health component under the direction of a parent management company as a subsidiary of the association;
providing the association health component with a health care service, an operational process, and a managing general agent who licenses the operational process and manages the operational process for the association health component;
managing the health care service for the association;
training the sales and administrative personal of the association health component;
providing a wellness service to the association health component under the direction of the parent management company;
providing a risk management service to the association health component under the direction of the parent management company;
providing a financial service to the association health component under the direction of the parent management company; and
providing a data management service to capture patient and employer for the association health plan component under the direction of the parent management company.

15. The method of claim 14, wherein association health component is a captive association health plan company.

16. The method of claim 14, wherein association health component is a workers compensation company and the health care service is a workers compensation insurance plan.

17. The method of claim 14, wherein the managing general agent is an independent contractor.

18. The method of claim 14, wherein the managing general agent is an employee of the association health component.

19. The method of claim 14, wherein the managing general agent is an employee of the parent management company.

20. The method of claim 14, further comprising providing access to the health care service, the wellness service, the risk management service, the association health component, the parent management company, and the data management service through an Internet component.
